(19) United States
(12) Patent Application Publication
(10) Pub. No.: US 2026/0036589 A1
(43) Pub. Date: Feb. 5, 2026

(54) METHODS FOR PREVENTING ARTIFICIAL DISULFIDE SCRAMBLING IN NON-REDUCED PEPTIDE MAPPING

(71) Applicant: Regeneron Pharmaceuticals, Inc., Tarrytown, NY (US)

(72) Inventors: Andrew Kleinberg, Roslyn Heights, NY (US); Yuan Mao, Hartsdale, NY (US)

(21) Appl. No.: 19/285,838

(22) Filed: Jul. 30, 2025

Related U.S. Application Data
(60) Provisional application No. 63/677,869, filed on Jul. 31, 2024.

(51) Int. Cl. G01N 33/68

(52) U.S. Cl. CPC G01N 33/6818; G01N 33/6851

(57) ABSTRACT

Methods for performing non-reduced peptide mapping analysis of a protein of interest can include alkylating a protein of interest with N-ethyl maleimide (NEM) or an NEM analog under denaturing conditions to form an alkylated protein of interest; digesting the alkylated protein of interest with at least one digestive enzyme to form a peptide digest, and subjecting the peptide digest to liquid-chromatography-mass spectrometry analysis. The methods can be performed under mildly alkaline conditions.

### CROSS-REFERENCE TO RELATED APPLICATION

This application claims the benefit of and priority to U.S. Provisional Application No. 63/677,869, filed 31 Jul. 2024, entitled “METHODS FOR PREVENTING ARTIFICIAL DISULFIDE SCRAMBLING IN NON-REDUCED PEPTIDE MAPPING”, the entire disclosure of which is hereby incorporated by reference herein.

### SEQUENCE LISTING

The instant application contains a Sequence Listing which is being submitted herewith electronically in XML format and is hereby incorporated by reference in its entirety. Said XML copy, created on 31 Jul. 2025, is named “086939502401_Sequence_Listing_2_ST26.xml” and is 66,422 bytes in size.

### TECHNICAL FIELD

The present disclosure generally pertains to methods for performing non-reduced peptide mapping of a protein of interest including alkylation, denaturing, digesting and analyzing by liquid chromatography-mass spectrometry to obtain a non-reduced peptide mapping of the protein of interest.

### BACKGROUND

Characterization of monoclonal antibodies' (mAbs) product quality attributes (PQAs) is important due to the large size and complex heterogeneity of this major class of therapeutics. One such PQA is the proper formation of classical disulfide bond structures. Deviations from the canonical IgG disulfide conformation, including non-classical disulfide bonding (scrambling), may negatively impact the structure, stability, and biological efficacy of a mAb.

Mass spectrometry (MS) has become an important technique for detailed protein analysis because of its ability to differentiate species with unique masses. The most common MS approach for characterizing disulfide connections between specific cysteines in a protein is non-reduced peptide mapping. In bottom-up MS-based proteomics, sample preparation steps, such as alkylation and digestion, are routine steps to facilitate peptide identification. However, conventional digestion strategies used throughout the biopharmaceutical industry have been shown to cause unintentional rearrangement of disulfide connections (disulfide scrambling), thus generating connectivity profiles that do not accurately represent the protein being analyzed.

Common misconceptions (e.g., avoiding basic-pH digestion to prevent disulfide scrambling) have led to the development of alternative reagents and conditions that can alleviate this issue. However, trypsin digestion under these conditions may result in non-specific or missed cleavages, yielding problematic digestion profiles. Therefore, methods are needed that can prevent the formation of sample preparation-induced disulfide scrambling and produce predictable digestion profiles.

Further, once a non-reduced peptide digest has been successfully prepared with minimal artificial disulfide scrambling, any scrambled disulfides detected can be assumed to have been present in the initial protein sample. However, disulfide peptides can often be difficult to characterize, particularly when analyzing low-abundance scrambled disulfides which have significantly weaker MS signal intensities. Therefore, methods are needed for the identification of disulfide bonds in a protein.

### SUMMARY

This disclosure provides a method for performing non-reduced peptide mapping analysis of a protein of interest in a sample while preventing the formation of sample preparation-induced disulfide scrambling. In an implementation, the method can comprise: (a) contacting a sample including the protein of interest to N-ethyl maleimide (NEM) or an NEM analog under denaturing conditions to form an alkylated sample; (b) contacting the alkylated sample to at least one digestive enzyme to form a peptide digest; and (c) subjecting the peptide digest to analysis using liquid chromatography-mass spectrometry to obtain the non-reduced peptide mapping of the protein of interest.

In another implementation, the method can comprise: (a) alkylating a protein of interest with N-ethyl maleimide (NEM) or an NEM analog under denaturing conditions to form an alkylated protein of interest; (b) digesting the alkylated protein of interest with at least one digestive enzyme to form a peptide digest; and (c) analyzing the peptide digest by liquid chromatography-mass spectrometry to obtain a non-reduced peptide mapping of the protein of interest. Advantageously, step (a) can include premixing a denaturing agent with either or both of the N-ethyl maleimide (NEM) or the NEM analog to form a premixture and adding the protein of interest to the premixture.

In one aspect, the methods can further comprise identifying disulfide bonds in the protein of interest.

In one aspect, the protein of interest is an antibody, a bispecific antibody, a monoclonal antibody, a fusion protein, an antibody-drug conjugate, an antibody fragment, or a protein pharmaceutical product.

In one aspect, the NEM analog is maleimide. In another aspect, the NEM analog is less hydrophobic than NEM. In another aspect, the NEM analog has a shorter retention time than NEM. In another aspect, the concentration of NEM or NEM analog in the sample used in the alkylation step can range from about 1 mM to about 10 mM and any value or range thereof such as from about 2 mM to about 8 mM, such as about 4 mM.

In one aspect, the sample is contacted with NEM or an NEM analog under denaturing conditions at a temperature and time sufficient for alkylation of free thiolson the protein of interest. For example, the sample is contacted with NEM or an NEM analog under denaturing conditions at a temperature from about 25° C. to about 65° C., such as at about 50° C.±10° C. and from about 1 minute to about 180 minutes, such as from about 5 minutes to 30 minutes, e.g., for 10 minutes±5 minutes. In another aspect, the denaturing conditions include use of urea, guanidinium chloride, organic solvents, or a combination thereof. In yet another aspect, the denaturing conditions include use of urea such as 8 M urea.

In one aspect, the alkylation and denaturation step can be conducted at acidic or alkaline pH. For example, the protein of interest can be subjected to alkylation and denaturation at a pH in the range of from about 5.0 to about 9.0, such as from about 6.5 to about 8.3, or any range or value thereof, such as at a pH of about 7.5±0.3.

In one aspect, the at least one digestive enzyme includes an immunoglobulin G-degrading enzyme of Streptococcus pyogenes, sialidase, cysteine protease, endopeptidase, papain, endoproteinase Lys-C, pepsin, trypsin, carboxypeptidase B, protease, or a combination thereof. In another aspect, the at least one digestive enzyme includes Lys-C, a trypsin, or a combination thereof.

In one aspect, the digestion is conducted at a pH from about 5 to about 8. In another aspect, digestion is conducted at a pH from about 7.0 to about 8.0. In yet another aspect, the digestion for step (a) is conducted at a pH of about 7.5±0.3, or 7.5±0.2.

In one aspect, the digestion is conducted at a temperature from about 20° C. to about 60° C., such as from about 25° C. to about 55° C. and any range or value thereof. Further, digesting an alkylated protein of interest with at least one digestive enzyme to form a peptide digest may be carried out for sufficient time to complete digestion of the protein of interest. In some exemplary embodiments, digestion can be carried out for up to and including about 12 hours, e.g., for a time period of 1 hour to 12 hours, such as from about 2 hours to 6 hours, or any range or values thereof.

In one aspect, analyzing the peptide digest by liquid chromatography-mass spectrometry includes analysis with reversed phase liquid chromatography, ion exchange chromatography, size exclusion chromatography, affinity chromatography, hydrophobic interaction chromatography, hydrophilic interaction chromatography, mixed-mode chromatography, or a combination thereof.

Advantageously, analyzing a peptide digest by liquid chromatography entails loading the peptide digest on to a chromatography support and eluting the peptide digest into its components using one or more mobile phase solutions in which an eluate including one or more components of the peptide digest are analyzed by mass spectrometry.

In an implementation, analyzing the peptide digest by liquid chromatography-mass spectrometry can include analysis with an liquid chromatography system coupled to or separate from a mass spectrometer. In one aspect, the mass spectrometer is an electrospray ionization mass spectrometer, nano-electrospray ionization mass spectrometer, quadrupole mass spectrometer, an Orbitrap-based mass spectrometer, or a hybrid quadrupole-Orbitrap-based mass spectrometer.

In one aspect, an amino acid is added to an eluate between the liquid chromatography and the mass spectrometer. In another aspect, the amino acid is glycine.

This disclosure provides a method for identifying scrambled disulfide bonds in a peptide digest, comprising (a) preparing the peptide digest, said preparing comprises (i) contacting a sample including the protein of interest to denaturing conditions to form a denatured sample, and; (ii) contacting the denatured sample to at least one digestive enzyme to form a peptide digest; and (b) subjecting the peptide digest to analysis using liquid chromatography-mass spectrometry to identify the scrambled disulfide bonds in the protein of interest.

In one aspect, the protein of interest is an antibody, a bispecific antibody, a monoclonal antibody, a fusion protein, an antibody-drug conjugate, an antibody fragment, or a protein pharmaceutical product.

In one aspect, the NEM analog is maleimide. In another aspect, the NEM analog is less hydrophobic than NEM. In another aspect, the NEM analog has a shorter retention time than NEM. In another aspect, the concentration of NEM or NEM analog used to contact the sample is about 4 mM.

In one aspect, the sample is contacted with NEM or an NEM analog under denaturing conditions at 50° C. for 10 minutes. In another aspect, the denaturing conditions include use of urea, guanidinium chloride, organic solvents, or a combination thereof. In yet another aspect, the denaturing conditions include use of 8 M urea.

In one aspect, the at least one digestives enzyme includes an immunoglobulin G-degrading enzyme of Streptococcus pyogenes, sialidase, cysteine protease, endopeptidase, papain, endoproteinase Lys-C, pepsin, trypsin, carboxypeptidase B, protease, or a combination thereof. In another aspect, the at least one digestives enzyme includes Lys-C, a trypsin, or a combination thereof.

In one aspect, the digestion is conducted at a pH of about 8.5. In another aspect, the digestion is conducted at a temperature of 37° C. In another aspect, the digestion is conducted for about 4 hours.

In one aspect, the chromatography step comprises reversed phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, the mass spectrometer is an electrospray ionization mass spectrometer, nano-electrospray ionization mass spectrometer, quadrupole mass spectrometer, an Orbitrap-based mass spectrometer, or a hybrid quadrupole-Orbitrap-based mass spectrometer, wherein the mass spectrometer is coupled to the liquid chromatography system. In another aspect, the mass spectrometry is tandem mass spectrometry.

In one aspect, an amino acid is added to the eluate between the liquid chromatography and the mass spectrometry. In another aspect, the amino acid is glycine.

The disclosure also provides a method for identifying native disulfide bonds in a protein of interest, comprising (a) performing non-reduced peptide mapping analysis of the protein of interest to identify disulfide bonds in a peptide digest of the protein of interest, the performing comprises: (i) contacting a sample including the protein of interest to N-ethyl maleimide (NEM) or an NEM analog under denaturing conditions to form an alkylated sample; (ii) contacting the alkylated sample to at least one digestive enzyme to form a peptide digest; and (iii) subjecting the peptide digest to analysis using liquid chromatography-mass spectrometry to identify the disulfide bonds in the peptide digest; (b) identifying scrambled disulfide bonds in the peptide digest, the identifying comprises: (i) contacting a sample including the protein of interest to denaturing conditions to form a denatured sample; (ii) contacting the denatured sample to at least one digestive enzyme to form a peptide digest; and (iii) subjecting the peptide digest to analysis using liquid chromatography-mass spectrometry to identify the scrambled disulfide bonds in the peptide digest; and comparing the identified disulfide bonds from non-reduced peptide mapping analysis to the identified scrambled disulfide bonds to identify the native disulfide bonds in the protein of interest.

In one aspect, the protein of interest is an antibody, a bispecific antibody, a monoclonal antibody, a fusion protein, an antibody-drug conjugate, an antibody fragment, or a protein pharmaceutical product.

In one aspect, the NEM analog is maleimide. In another aspect, the NEM analog is less hydrophobic than NEM. In another aspect, the NEM analog has a shorter retention time than NEM. In another aspect, the concentration of NEM or NEM analog used to contact the sample is about 4 mM.

In one aspect, the sample is contacted with NEM or an NEM analog under denaturing conditions in step (a) at 50° C. for 10 minutes. In another aspect, the sample is contacted to denaturing conditions in step (b) at 50° C. for 10 minutes. In another aspect, the denaturing conditions include use of urea, guanidinium chloride, organic solvents, or a combination thereof. In yet another aspect, the denaturing conditions include use of 8 M urea.

In one aspect, the at least one digestives enzyme includes an immunoglobulin G-degrading enzyme of Streptococcus pyogenes, sialidase, cysteine protease, endopeptidase, papain, endoproteinase Lys-C, pepsin, trypsin, carboxypeptidase B, protease, or a combination thereof. In another aspect, the at least one digestives enzyme includes Lys-C, a trypsin, or a combination thereof.

In one aspect, the digestion for step (b) is conducted at a pH from about 5 to about 8. In another aspect, the digestion for step (b) is conducted at a pH from about 7 to about 8. In yet another aspect, the digestion for step (b) is conducted at a pH of about 7.5±0.3, or 7.5±0.2.

In one aspect, the digestion for step (a) and (b) is conducted at a temperature of about 37° C. In another aspect, the digestion for step (a) and (b) is conducted for about 4 hours.

In one aspect, the chromatography step comprises reversed phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, the mass spectrometer is an electrospray ionization mass spectrometer, nano-electrospray ionization mass spectrometer, quadrupole mass spectrometer, an Orbitrap-based mass spectrometer, or a hybrid quadrupole-Orbitrap-based mass spectrometer, wherein the mass spectrometer is coupled to the liquid chromatography system. In another aspect, the mass spectrometry is tandem mass spectrometry.

In one aspect, an amino acid is added to the eluate between the liquid chromatography and the mass spectrometry. In another aspect, the amino acid is glycine.

Additional advantages of the present disclosure will become readily apparent to those skilled in the art from the following detailed description, wherein only certain embodiments are shown and described, simply by way of illustration of carrying out certain subject matter. As will be realized, the invention is capable of other and different embodiments, and its several details are capable of modifications in various respects, all without departing from the invention. Accordingly, the drawings and description are to be regarded as illustrative in nature, and not as restrictive.

### BRIEF DESCRIPTION OF THE DRAWINGS

FIG. 1 shows UV chromatograms (215 nm) of non-reduced tryptic digests of cemiplimab under various conditions. Methods A, B and C involve digestion at pH 7.5 with various alkylation reagents (iodoacetamide, N-ethyl maleimide (NEM), and no alkylation, respectively). Methods D and E involve digestion at pH 5.3 with Promega's AccuMAP Low pH Protein Digestion Kit with NEM and no alkylation, respectively. Trypsin missed cleavages, non-specific cleavages and protease autolysis peaks are labeled with an asterisk (*).

FIG. 2 shows a relative MS response heat map for all possible unique scrambled disulfide connections within cemiplimab after trypsin digestion under various conditions and alkylating reagents. Digestions were performed at 37° C. for 4 h. All major charge states and isotopic peaks were included in the EIC peak integration. “Not Detected” indicates that the disulfide peptide was not identified even in the positive control digests with no alkylation (methods C and F).

FIG. 3 shows MS responses for a common IgG4 mAb cysteine-containing tryptic peptide alkylated with either NEM or iodoacetamide (IAM), as well as a scrambled disulfide involving the same peptide. Samples of cemiplimab were denatured, alkylated and digested under non-reducing conditions at pH 7.5. All major charge states and isotopic peaks were included in the EIC peak integrations.

FIG. 4 illustrates a generic scheme of possible disulfide decomposition mechanism due to β-elimination. Formation of free cysteine from native disulfide peptides can lead to formation of artificial disulfide scrambling.

FIG. 5 shows MS responses for scrambled disulfides in tryptic digests of cemiplimab under variable pH and time. All samples were initially alkylated with NEM to ensure that any significant scrambling formation would be due to alternative mechanisms such as β-elimination. All major charge states and isotopic peaks were included in the EIC peak integrations.

FIG. 6 shows UV absorbance at 215 nm of various cysteine-alkylating reagents after incubation for 4 h at pH 7.5, according to an exemplary embodiment.

FIG. 7 shows UV chromatograms of non-reduced peptide mapping trypsin digests of cemiplimab (method B) using NEM and maleimide, respectively, for cysteine alkylation, according to an exemplary embodiment. The chromatogram of the digest containing NEM shows two large reagent peaks, potentially obstructing peptide UV peaks. The chromatogram of the digest containing maleimide has no reagent peaks within the peptide elution time window (5-80 min).

### DETAILED DESCRIPTION

Monoclonal antibodies (mAbs) have become an increasingly popular form of therapeutics for a wide range of illnesses, including cancer, autoimmune disorders and infectious diseases. mAbs are distinguished from traditional small molecule medications by their specificity toward designed targets, owing to their complex 3D structures, which are maintained through covalent disulfide bonds between cysteine residues. With improper disulfide connectivity (disulfide scrambling), proteins may exhibit incorrect folding and stability issues, and become unable to bind the intended antigens. Correct disulfide linkage is therefore a critical quality attribute of mAbs. However, ensuring the completeness of this correct linkage can be analytically challenging.

Mass spectrometry (MS) is currently the most practical technique for detailed protein analysis because of its ability to differentiate species with unique masses. Many common post-translational modifications in mAbs can be identified through both top-down (intact MS) and bottom-up (peptide mapping) approaches because the native and modified forms possess different masses. However, intra-protein disulfide scrambling cannot be distinguished from proper disulfide connectivity through top-down MS analysis because no overall difference in mass exists between variants with rearranged disulfide bonds. The preferred method to completely and explicitly characterize local disulfide connectivity is a bottom-up peptide approach under non-reducing conditions. Unfortunately, performing mAb digestions under traditional non-reducing conditions has been extensively demonstrated to rearrange native disulfide connections, thus inducing new artificial disulfide scrambling, which cannot be distinguished from similar scrambling present within the undigested protein sample in the data analysis. Thus, in order to limit disulfide rearrangement, detailed knowledge of the mechanisms underlying artificial disulfide scrambling is required.

Furthermore, because the levels of disulfide scrambling, whether pre-existing or digest-induced, are often low within mAb digests, the detection and identification of each peptide can be challenging even with a sensitive mass spectrometer. Previous work by the inventors described a simple method for confident detection and identification of nearly all possible disulfide scrambling permutations for a given mAb, in which test samples were compared against a positive control digest of the same mAb with purposefully induced disulfide scrambling (Andrew Kleinberg, Rachel Joseph, Yuan Mao, and Ning Li. Ultrasensitive disulfide scrambling analysis of mAbs by LC-MS with post-column reduction and glycine signal enhancement. Anal Biochem. 2022; 653, 114773).

Herein, a markedly improved positive control digest that exploits multiple combined principles of artificially induced disulfide scrambling is described. In addition, the inventor's recent discovery of glycine as a TFA mobile phase additive has been shown to boost the MS signal intensity of peptides by more than an order of magnitude on average, without compromising chromatography (Yuan Mao, Andrew Kleinberg, Yunlong Zhao, Shivkumar Raidas S, and Ning Li. Simple addition of glycine in trifluoroacetic acid-containing mobile phases enhances the sensitivity of electrospray ionization mass spectrometry for biopharmaceutical characterization. Anal Chem. 2020, 92(13), 8691-96). These combined techniques were applied to the data reported herein to increase the confidence of identifying disulfide scrambling within mAb digests.

Due to the labile reactivity of the sulfhydryl group (—SH) of cysteine, alkylation prior to digestion is the preferred method to minimize numerous side-reactions (e.g., oxidation and disulfide scrambling). Current popular sulfhydryl alkylating reagents contain iodo-substituting groups such as iodoacetamide and iodoacetic acid, which react with free cysteine via the S_{N}2 nucleophilic substitution mechanism. These reagents have the benefit of high-selectivity toward sulfhydryls and room temperature reactivity, although they are generally limited to basic-pH conditions, may over-alkylate under certain conditions or decompose when exposed to light. Thus, they are excellent choices for reduced peptide mapping of mAbs, because all disulfide bonds are intentionally broken, and complete alkylation can be easily achieved.

For non-reduced peptide mapping, cysteine alkylation is recommended because native mAb samples commonly contain low levels of unbonded free thiol cysteines (because of post-translational factors). Therefore, cysteines in mAb should ideally be fully paired and disulfide bonded to maintain the intended 3D structure to perform biological functions. Free thiols shielded within the folded structure cannot be alkylated without first denaturing the mAb to increase solvent-accessibility. However, after denaturation, the free thiols gain movement flexibility and consequently can approach and displace native disulfide connections through a thiol-disulfide exchange mechanism, thereby forming new disulfide connections not present in the original protein sample (i.e., disulfide scrambling). Subsequent digestion would then generate a peptide map that is not fully representative of the protein being analyzed. To minimize this phenomenon, an alkylating reagent should be chosen that can react with free thiols faster than disulfide rearrangement can occur. Previous reports have indicated that iodo-based reagents fall short in this regard (Volker Schnaible et al., Partial reduction and two-step modification of proteins for identification of disulfide bonds. Anal Chem. 2002, 74(10), 2386-93).

Another common cysteine alkylating reagent is N-ethyl maleimide (NEM), which reacts with free thiols via the Michael addition mechanism. The flat maleimide ring structure is a particularly reactive Michael acceptor because of the strain caused by its conjugated double bonds, which restrict rotation into a more relaxed state. Thiol-maleimide coupling is often referred to as “click” chemistry because of its speed, convenience and efficiency. NEM can alkylate cysteine under a wide pH range (pH 3-9), although at basic pH, it may begin to react with amino groups (e.g., lysine side chain and peptide N-terminus). Using NEM under acidic-pH conditions has been reported to prevent artificial disulfide scrambling in non-reduced mAb digests (Shan Lu et al., Mapping native disulfide bonds at a proteome scale. Nat Methods. 2015, 12, 329-31). Previous work by the inventors has detailed the profound extensiveness of these effects in digestions comparing NEM and iodoacetamide (Andrew Kleinberg et al., Anal Biochem. 2022, 653, 114773). Unfortunately, despite the utility of commercial products such as Promega's AccuMAP Low pH Protein Digestion Kit, performing tryptic digests under acidic conditions is not ideal because many non-specific or missed cleavages may occur, often yielding crowded and messy chromatograms.

The disclosure herein provides an investigation into individual factors contributing to artificial disulfide scrambling. Through the analyses of these factors, a method for non-reduced peptide mapping of a protein of interest is provided. The methods of the present disclosure can provide an efficient non-reduced digestion protocol to generate a simple, predictable peptide map with an accurate disulfide connectivity profile. Further, a method is provided to intentionally maximize disulfide scrambling as an identification tool and reference.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included. As used herein, the terms “include,” “includes,” and “including” are meant to be non-limiting and are understood to mean “comprise,” “comprises,” and “comprising” respectively.

As used herein, the term “sample” refers to a mixture of molecules comprising a protein of interest, that is subjected to manipulation in accordance with the methods of the present disclosure, including, for example, separating, analyzing, extracting, concentrating, profiling and the like. A sample can be obtained from any step of a bioprocess, such as cell culture fluid (CCF), harvested cell culture fluid (HCCF), any step in the downstream processing, drug substance (DS), or a drug product (DP) comprising the final formulated product.

As used herein, the term “protein” or “protein of interest” includes any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides' refers to a non-naturally occurring peptide or polypeptide. Synthetic peptides or polypeptides can be synthesized, for example, using an automated polypeptide synthesizer. Various solid phase peptide synthesis methods are known. A protein may contain one or multiple polypeptides to form a single functioning biomolecule. A protein can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a review discussing biotherapeutic proteins and their production, see Ghaderi et al., “Production platforms for biotherapeutic glycoproteins. Occurrence, impact, and challenges of non-human sialylation,” (BIOTECHNOL. GENET. ENG. REV. 147-175 (2012)). In some exemplary embodiments, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H2} and C_{H}3. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different exemplary embodiments, the FRs of the anti-big-ET-1 antibody (or antigen-binding portion thereof) may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex.

The term “bispecific antibody” (bsAbs) includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, the term “disulfide bond” refers to a covalent bond derived from two thiol groups. In proteins, such as for example monoclonal antibodies, these bonds from between the thiol groups of two cysteine amino acids. Disulfide bonds contribute to stabilizing protein globular structure and holding proteins in their respective conformation, thus having an important role in protein folding and stability. As discussed herein, a disulfide, or disulfide peptide, encompasses two peptides covalently bonded though cysteine residues on each corresponding peptide, and each of the two peptides in their reduced form are referred to as “reduced partner peptides.” Such disulfide peptides can be generated via protease digestion (e.g., trypsin protease digestion and/or recombinant Lys-C protease digestion) under non-reducing conditions, where disulfide bonds remain intact. Such disulfide peptides can then be reduced to their corresponding reduced partner peptides via a reducing agent (e.g., DTT, TCEP, etc.).

As used herein, the term “scrambled disulfide” or “disulfide scrambling” encompasses disulfide bonds that are non-native to a particular biomolecule, such as a monoclonal antibody.

In some exemplary embodiments, the sample comprising the protein of interest can be subjected to alkylation, denaturation, and/or digestion.

As used herein, the term “protein alkylating agent” or “alkylation agent” refers to an agent used for alkylating certain free amino acid residues in a protein. Non-limiting examples of commercial protein alkylating agents are iodoacetamide (IOA/IAA), chloroacetamide (CAA), acrylamide (AA), N-ethylmaleimide (NEM), maleimide, methyl methanethiosulfonate (MMTS), and 4-vinylpyridine, any analogs thereof, or combinations thereof.

In some exemplary embodiments, the alkylation agent used is NEM or a NEM analog, such as maleimide. NEM or maleimide can be used at a relatively wide range of concentrations for alkylating a protein of interest. The concentration of NEM or a NEM analog, e.g., maleimide may be from about 1 mM to about 10 mM and any value or range thereof. For example, the concentration of NEM or maleimide may range from about 1 mM to about 8 mM and may be about 1 mM, about 1.1 mM about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, about 3 mM, about 3.1 mM, about 3.2 mM, about 3.3 mM, about 3.4 mM, about 3.5 mM, about 3.6 mM, about 3.7 mM, about 3.8 mM, about 3.9 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM. In some exemplary embodiments, a concentration of NEM or maleimide is about 4 mM.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof. In an aspect of the present disclosure, denaturation agents can include guanidine hydrochloride and/or urea. In some exemplary embodiments, the denaturation agent is urea.

In certain exemplary embodiments, the methods of the present disclosure do not include di-sulfide reducing agents such as dithiothreitol (DTT), β-mercaptoethanol, TCEP (tris(2-carboxyethyl)phosphine).

A denaturing agent, such as urea, can be included in the alkylation step to form denaturing conditions. The denaturing agent can be included at a concentration of from about 5M to about 10 M and any value or range thereof. For example, the concentration of denaturing agent, e.g., urea, may range from about 6 M to about 8 M and may be about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, about 8 M, about 8.1 M, about 8.2 M, about 8.3 M, about 8.4 M, about 8.5 M, about 8.6 M, about 8.7 M, about 8.8 M, about 8.9 M, about 9 M, about 9.1 M, about 9.2 M, about 9.3 M, about 9.4 M, about 9.5 M, about 9.6 M, about 9.7 M, about 9.8 M, about 9.9 M, or about 10 M. In some exemplary embodiments, a concentration of the denaturing agent, e.g., urea, is about 8 M.

To facilitate rapid alkylation of the protein of interest, the denaturing agent and the N-ethyl maleimide (NEM) or the NEM analog can be premixed to form a premixture prior to adding the protein of interest to the premixture.

In some exemplary embodiments, the sample comprising the protein of interest can be subjected to alkylation and denaturation at acidic or alkaline pH (e.g, from about 5.0 to about 9.0). For example, the protein of interest can be subjected to alkylation and denaturation at a pH in the range of from about 5.0 to about 7.0, such as from about 6.0 to about 7.0, e.g., from about 6.5 to about 7.0 as an acidic pH, or from about 7.0 to about 9.0, such as from about 7.0 to about 8.0, e.g., about a pH of 7.5±0.3 as an alkaline pH. Further, the alkylation and denaturation may be conducted at a pH of about 5, about 5.1, about 5.2, about 5.3, about 5.4, about 5.5, about 5.6, about 5.7, about 5.8, about 5.9, about 6.0, about 6.1, about 6.2, about 6.3, about 6.4,, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.5, or about 9.0. In some exemplary embodiments, a pH for alkylation and/or denaturation is about 7.5±0.3, or about 7.5±0.2. In some exemplary embodiments, an optimal pH for alkylation and/or denaturation is about 8.5.

Alkylation and denaturation may be carried out in ambient (room) temperature or above ambient temperature. In some exemplary embodiments, alkylation and denaturation is carried out at a temperature from about 20° C. to about 60° C., such as from about 25° C. to about 55° C. and any range or value thereof, such as at a temperature at about 25° C., about 37° C., about 45° C., about 50° C., or about 55° C. The mixture may be incubated for a period of time in order to ensure complete denaturation and alkylation of the protein of interest. In some exemplary embodiments, the mixture may be incubated for about 2 minutes, about 3 minutes, about 4 minutes, about 5 minutes, about 6 minutes, about 7 minutes, about 8 minutes, about 9 minutes, about 10 minutes, about 15 minutes, about 30 minutes, about 45 minutes, about 1 hour, about 1.5 hour, or about 2 hours.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

Digestive enzymes used for peptide mapping may include, for example, one or more of trypsin, pepsin, or LysC. In some exemplary embodiments, the digestive enzyme is trypsin. The digestive enzyme, such as trypsin may be used at an enzyme: substrate ratio (E/S) of about 1:5, about 1:5.5, about 1:6, about 1:6.5, about 1:7, about 1:7.5, about 1:8, about 1:8.5, about 1:9, about 1:9.5, about 1:10, about 1:10.5, about 1:11, about 1:11.5, about 1:12, about 1:12.5, about 1:13, about 1:13.5, about 1:14, about 1:14.5, about 1:15, about 1:16, about 1:17, about 1:18, about 1:19, about 1:20, about 1:25, about 1:28, etc. and any range or value therebetween. In some exemplary embodiments, an optimal enzyme: substrate ratio of trypsin from about 1:10 to about 1:22.

Digestion, such as digestion of an alkylated protein of interest, may be conducted at a pH in a range of from about 5.0 to about 9.0, or any range or value thereof, such as from about 6.0, about 6.1, about 6.2, about 6.3, about 6.4, about 6.5, about 6.6, about 6.7, about 6.8, about 6.9, about 7.0 to about 8.5, about 8.4, about 8.4, about 8.3, about 8.2, about 8.1, about 8.0, etc. and any range or value thereof, e.g., from about 6.5 to about 8.3 or from about 7.0 to about 8.0. Further, digestion, such as digestion of an alkylated protein of interest, may be conducted at a pH of about 5, about 5.1, about 5.2, about 5.3, about 5.4, about 5.5, about 5.6, about 5.7, about 5.8, about 5.9, about 6.0, about 6.1, about 6.2, about 6.3, about 6.4,, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.5, or about 9.0. In some exemplary embodiments, a pH for digestion is about 7.5±0.3, or 7.5±0.2.

Digestion may be carried out in ambient (room) temperature or above ambient temperature. In some exemplary embodiments, digestion is carried out at a temperature from about 20° C. to about 60° C., such as from about 25° C. to about 55° C. and any range or value thereof, such as at a temperature of about 25° C., about 37° C., about 45° C., or about 55° C. Further, digesting an alkylated protein of interest with at least one digestive enzyme to form a peptide digest may be carried out for sufficient time to complete digestion of the protein of interest. In some exemplary embodiments, digestion can be carried out for up to and including about 12 hours, such as from up to about 1 hour, about 2 hours, about 3 hours, about 4 hours, about 5 hours, about 6 hours, about 7 hours, about 8 hours, about 9 hours, about 10 hours and up to and including 12 hours. In some specific exemplary embodiments, digestion can be carried out for a time period of 1 hour to 12 hours, such as from about 2 hours to 6 hours, or any range or values thereof.

In some specific exemplary embodiments, the sample can be selected from any step of the downstream process of clarification, chromatographic production, or filtration that is used in preparing the protein of interest.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase liquid chromatography, ion-exchange chromatography, size exclusion chromatography, affinity chromatography, hydrophobic interaction chromatography, hydrophilic interaction chromatography, or mixed-mode chromatography. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection. Analyzing a peptide digest by liquid chromatography entails loading the peptide digest on to a chromatography support and eluting the peptide digest into its components using one or more mobile phase solutions in which an eluate including one or more components of the peptide digest are analyzed by a mass spectrometry.

As used herein, the term “mass spectrometry” includes the use of a device capable of identifying specific molecular species and measuring their accurate masses. The term is meant to include any molecular detector into which an oligonucleotide may be characterized. The mass spectrometer can be coupled to a liquid chromatography system, wherein the mass spectrometer is capable of performing LC-MS (liquid chromatography-mass spectrometry). A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. Non-limiting examples of ion sources include electrospray ionization (ESI), atmospheric pressure ionization (API), matrix assisted laser desorption ionization (MALDI), laser desorption ionization (LDI), and desorption electrospray ionization (DESI). The term “mass analyzer” refers to a device that can separate species, that is, atoms, molecules, or clusters, according to their mass. Non-limiting examples of mass analyzers include time-of-flight (TOF), magnetic/electric sector, quadrupole mass filter (Q), quadrupole ion trap (QIT), orbitrap, Fourier transform ion cyclotron resonance (FTICR), and also the technique of accelerator mass spectrometry (AMS).

In some exemplary embodiments, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information or the fragment ion signal is detectable. Tandem MS have been successfully performed with a wide variety of analyzer combinations. What analyzers to combine for a certain application is determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization can include, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

It is understood that the present disclosure is not limited to any of the aforesaid sample(s), protein(s), protein(s) of interest, antibody(s), antibody fragment(s), protein alkylating agent(s), protein denaturing agent(s), digestive enzyme(s), liquid chromatograph(ies), mass spectrometer(s), tandem mass spectrometer(s), database(s), bioinformatics tool(s), pH, temperature(s), or concentration(s), and any protein(s), protein(s) of interest, antibody(s), antibody fragment(s), protein alkylating agent(s), protein denaturing agent(s), digestive enzyme(s), liquid chromatograph(ies), mass spectrometer(s), tandem mass spectrometer(s), database(s), bioinformatics tool(s), pH, temperature(s), or concentration(s) can be selected by any suitable means.

Various publications, including patents, patent applications, published patent applications, accession numbers, technical articles and scholarly articles are cited throughout the specification. Each of these cited references is herein incorporated by reference, in its entirety and for all purposes.

The disclosure will be more fully understood by reference to the following Examples, which are provided to describe the disclosure in greater detail. They are intended to illustrate examples and should not be construed as limiting the scope of the disclosure.

### EXAMPLES

Materials. Cemiplimab (IgG4, purity>99%, MW 143.8 kDa) was obtained from Regeneron (Tarrytown, NY). Urea, iodoacetamide, N-ethylmaleimide (NEM) and maleimide were purchased from Sigma (St. Louis, MO). Ultrapure 1.0 M Tris-HCl (pH 7.5 and 8.0) were purchased from Invitrogen Life Technologies (Carlsbad, CA). 1.0 M Tris HCl (pH 8.5) was purchased from Rockland (Limerick, PA). Sequencing grade modified trypsin and AccuMAP Low pH Protein Digestion Kit were purchased from Promega (Madison, WI). Trifluoroacetic acid (TFA, sequencing grade) and acetonitrile (Optima LC/MS) were purchased from Thermo Fisher Scientific (Waltham, MA). Glycine Ultrapure Bioreagent was purchased from J. T. Baker (Phillipsburg, NJ). High purity water was purchased from MilliporeSigma (Bedford, MA; Milli-Q system).

Non-reduced digestion of cemiplimab. Multiple non-reduced digests of cemiplimab were performed in parallel, each with slightly different conditions chosen to demonstrate the effects on the resulting disulfide scrambling profile. Generally, 100 μg of cemiplimab was denatured with 8 M urea in 0.1 M Tris-HCl (pH 7.5) at 50° C. for 10 min, then digested with trypsin (1:20 E/S) at 37° C. for 4 h after dilution of the urea concentration to 1.6 M with additional 0.1 M Tris-HCl (pH 7.5), and was finally quenched to pH 2 with TFA to stop trypsin digestion. During the initial denaturing step, the urea solution was pre-mixed with the chosen alkylating reagent, comprised of either 4 mM iodoacetamide (method A), 4 mM NEM (method B), or no alkylation reagent (method C).

Additional digests were performed according to the recommended procedure from Promega's AccuMAP Low pH Protein Digestion Kit, which was specifically designed to prevent disulfide scrambling and minimize other digestion-induced post-translational modifications. Generally, 50 μg of cemiplimab was denatured with 8 M urea in 1.4× AccuMAP Low pH Reaction Buffer at 37° C. for 30 min, followed by a pre-digestion step using AccuMAP Low pH Resistant rLys-C at 37° C. for 1 h, and the remaining digestion with AccuMAP Modified Trypsin and additional AccuMAP Low pH Resistant rLys-C was performed at 37° C. for 3 h (pH 5.3). Finally, digestion was quenched with TFA. One sample contained 8 mM NEM during the urea denaturing step (method D), and the other contained no alkylating reagent (method E). An additional 100-μg sample was denatured with 8 M urea in 0.1 M Tris-HCl (pH 8.5) at 50° C. for 10 min without alkylation, then digested with trypsin (1:20 E/S) at 37° C. for 4 h after dilution of the urea concentration to 1.6 M with additional 0.1 M Tris-HCl (pH 8.5), and was finally quenched to pH 2 with TFA (method F).

Peptide mapping analysis of cemiplimab. For peptide mapping analysis of cemiplimab, 5-μg aliquots of each tryptic digest were injected onto an ACQUITY UPLC system (Waters) equipped with a peptide BEH C18 column (Waters, 2.1 mm×150 mm, 1.7 μm particle size, 130 Å pore size). Peptide elution was subsequently performed with a linear gradient that increased from 0.1% mobile phase B to 40% mobile phase B over 80 min at a flow rate of 0.25 mL/min with a column temperature of 40° C. Mobile phase A consisted of 0.05% TFA in water, and mobile phase B consisted of 0.05% TFA in acetonitrile. All eluted peptides were monitored at a wavelength of 215 nm with a photodiode array detector. A mixing tee was set up to combine the post-column LC flow with a 0.1 M glycine solution, administered with a syringe pump at 5 μL/min (2 mM final glycine concentration). The resulting flow was electro-sprayed and analyzed with a Thermo Q-Exactive Plus hybrid mass spectrometer.

### Example 1. Comparison of Non-Reduced Digestion Protocols

Multiple non-reduced trypsin digests of cemiplimab (described in “Non-reduced digestion of cemiplimab”) were performed using common cysteine-alkylating reagents, as well as digests with no alkylation to purposefully maximize the occurrence of disulfide scrambling through the thiol-disulfide exchange mechanism. Basic-pH digestion without alkylation (method C) was effectively used as a positive control by forming nearly all possible scrambled disulfide permutations of cemiplimab, thereby generating each artificial peptide in relatively high abundance and enabling easy analysis of the traditional alkylated digest samples (methods A and B) through alignment of extracted ion chromatograms (EICs). The acidic-pH digest alkylated with NEM (method D) was used as a negative control for disulfide scrambling, because those conditions were demonstrated to prevent such artifacts in our previous study. In addition, to establish that the native cemiplimab protein indeed contained the free thiols required for the thiol-disulfide exchange scrambling mechanism, we report the observed MS responses of the alkylated cysteine-containing peptides from method B (Table 1).

TABLE 1
MS responses of NEM-alkylated cysteine-containing
peptides from a non-reduced peptide
mapping trypsin digest of cemiplimab (method B).

| | SEQ | | EIC |
|---|---|---|---|
| Cysteine- | ID | mAb | Peak |
| Containing Peptide | NO: | Domain | Area |
| LSCAASGFTFSNFGMTWVR | 1 | VHa | 5.42E+07 |
| GEDTAVYYCVK | 2 | VHb | 6.80E+06 |
| STSESTAALGCLVK | 3 | CH1a | 5.46E+08 |
| TYTCNVDHKPSNTK | 4 | CH1b | 1.01E+08 |
| TPEVTCVVVDVSQEDPEVQ | 5 | CH2a | 2.93E+08 |
| FNWYVDGVEVHNAK | | | |
| CK | 6 | CH2b | 8.19E+07 |
| NQVSLTCLVK | 7 | CH3a | 1.58E+09 |
| WQEGNVFSCSVMHEALHNH | 8 | CH3b | 3.48E+08 |
| YTQK | | | |
| DIQMTQSPSSLSASVGDSI | 9 | VLa | 7.00E+06 |
| TITCR | | | |
| TLQPEDFATYYCQQSSNTP | 10 | VLb | 7.44E+07 |
| FTFGPGTVVDFR | | | |
| SGTASVVCLLNNFYPR | 11 | CLa | 2.14E+08 |
| VYACEVTHQGLSSPVTK | 12 | CLb | 2.92E+08 |
| GPSVFPLAPCSR | 13 | HCLCa | 1.25E+08 |
| GEC | 14 | HCLCb | 1.54E+06 |

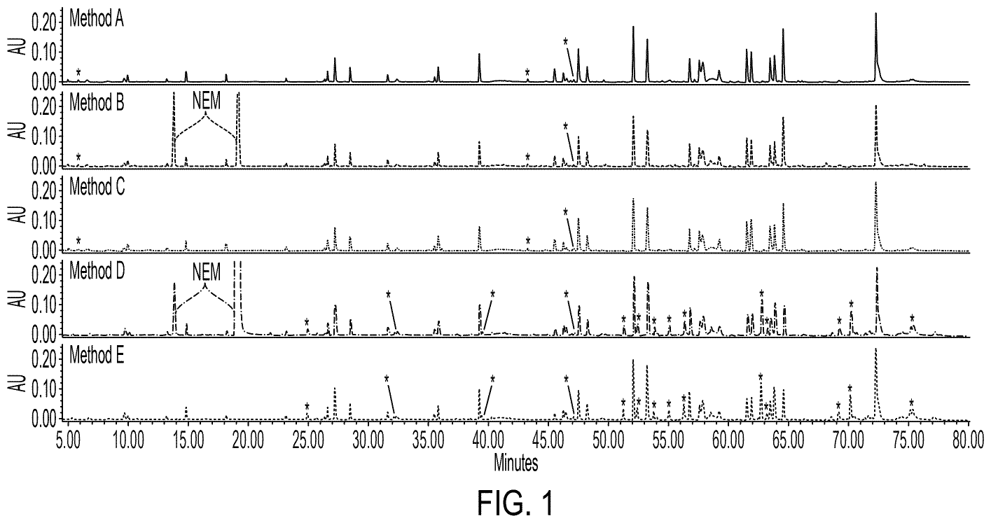

The UV chromatograms of the digests described above are shown in FIG. 1. All digests performed at pH 7.5 produced near-identical peak profiles despite the different alkylation choices, demonstrating that consistent tryptic peptide maps can be generated with various reagent classes under non-reduced conditions. All major UV peaks represented desired and predictable tryptic peptides, with only a few minor missed cleavage peptides (Table 2), thus indicating the excellent quality of the chosen conditions. The digests performed under acidic conditions (pH 5.3) with Promega's AccuMAP reagents and recommended protocol revealed many of the same peptides as the basic-pH digests, but were accompanied by several major tryptic missed cleavage peptides and possible enzyme autolysis peptides (Table 3), yielding more complex chromatograms because of the inefficiency of the digestive enzymes at this pH.

TABLE 2
Peptide identities of all major UV peaks from various non-reduced
peptide mapping trypsin digests of cemiplimab performed under
basic conditions (FIG. 1, methods A-C). All peptides were confirmed
using accurate full-MS and MS/MS analysis.

| Retention | | | | SEQ | |
|---|---|---|---|---|---|
| Time | Peptide | Disulfide | Peptide | ID | |
| (min) | Identity | Bond | Sequence | NO: | Comments |
| 5.0 | H215- | | VESK | 15 | |
| | 218 | | | | |
| 5.8 | H211- | | VDKR | 16 | missed |
| | 214 | | | | cleavage |
| 6.6 | H286- | | TKPR | 17 | |
| | 289 | | | | |
| 8.3 | H338- | | GQPR | 18 | |
| | 341 | | | | |
| 9.7 | H39- | | QAPGK | 19 | |
| | 43 | | | | |
| 10.0 | H315- | | EYK | 20 | |
| | 317 | | | | |
| 13.2 | H332- | | TISK | 21 | |
| | 335 | | | | |
| 14.8 | L184- | | ADYEK | 22 | |
| | 188 | | | | |
| 18.2 | L208- | | SFNR | 23 | |
| | 211 | | | | |
| 23.2 | H407- | | LTVDK | 24 | |
| | 411 | | | | |
| 26.6 | H290- | | EEQFN*STYR | 25 | glycopeptides |
| | 298 | | | | |
| 27.2 | L146- | | VQWK | 26 | |
| | 149 | | | | |
| 28.5 | L150- | | VDNALQSGNSQESVTEQDS | 27 | |
| | 169 | | K | | |
| 31.6 | H68- | | FTISR | 28 | |
| | 72 | | | | |
| 32.4 | H324- | | GLPSSIEK | 29 | |
| | 331 | | | | |
| 35.5 | H246- | | DTLMISR | 30 | |
| | 252 | | | | |
| 35.8 | H57- | | DTYFADSVK | 31 | |
| | 65 | | | | |
| 39.3 | H134- | C144H- | STSESTAALGCLVK | 32 | |
| | 147 | C200H | | | |
| | H197- | | TYTCNVDHKPSNTK | 33 | |
| | 210 | | | | |
| 41.0 | H342- | | EPQVYTLPPSQEEMTK | 34 | |
| | 357 | | | | |
| 43.3 | H77- | | NTLYLQMN | 35 | non-specific |
| | 84 | | | | cleavage |
| 45.5 | H122- | C131H- | GPSVFPLAPCSR | 36 | |
| | 133 | C214L | | | |
| | L212- | | GEC | 37 | |
| | 214 | | | | |
| 46.3 | L170- | | DSTYSLSSTLTLSK | 38 | |
| | 183 | | | | |
| 46.5 | H437- | | SLSLSLG | 39 | C-terminal |
| | 443 | | | | lysine loss |
| 46.9 | H122- | C131H- | GPSVFPLAPCSR | 40 | trisulfide |
| | 133 | C214L | | | |
| | L212- | | GEC | 41 | |
| | 214 | | | | |
| 47.1 | L150- | | VDNALQSGNSQESVTEQDS | 42 | missed |
| | 183 | | KDSTYSLSSTLTLSK | | cleavage |
| 47.5 | H44-56 | | GLEWVSGISGGGR | 43 | |
| | H77-87 | | NTLYLQMNSLK | 44 | |
| 48.2 | L62-76 | | FSGSGSGTDFTLTIR | 45 | |
| 52.1 | H358- | C364H- | NQVSLTCLVK | 46 | |
| | 367 | C422H | | | |
| | H414- | | WQEGNVFSCSVMHEALHN | 47 | |
| | 436 | | HYTQK | | |
| 53.3 | H1-19 | | EVQLLESGGVLVQPGGSLR | 48 | |
| | H368- | | GFYPSDIAVEWESNGQPEN | 49 | |
| | 389 | | NYK | | |
| 56.8 | H390- | | TTPPVLDSDGSFFLYSR | 50 | |
| | 406 | | | | |
| 57.6 | H253- | C258H- | TPEVTCVVVDVSQEDPEVQ | 51 | |
| | 285 | C318H | FNWYVDGVEVHNAK | | |
| | H318- | | CK | 52 | |
| | 319 | | | | |
| 57.9 | L127- | C134L- | SGTASVVCLLNNFYPR | 53 | |
| | 142 | C194L | | | |
| | L191- | | VYACEVTHQGLSSPVTK | 54 | |
| | 207 | | | | |
| 58.5 | L109- | | TVAAPSVFIFPPSDEQLK | 55 | |
| | 126 | | | | |
| 59.2 | L43-61 | | APNLLIYAASSLHGGVPSR | 56 | |
| 61.5 | H20-38 | C22H- | LSCAASGFTFSNFGMTWVR | 57 | |
| | H88-98 | C96H | GEDTAVYYCVK | 58 | |
| 61.9 | L25-42 | | ASLSINTFLNWYQQKPGK | 59 | |
| 63.5 | H99-121 | | WGNIYFDYWGQGTLVTVSS | 60 | |
| | | | ASTK | | |
| 63.9 | H299- | | VVSVLTVLHQDWLNGK | 61 | |
| | 314 | | | | |
| 64.6 | L1-24 | C23L- | DIQMTQSPSSLSASVGDSITI | 62 | |
| | | C88L | TCR | | |
| | L77-107 | | TLQPEDFATYYCQQSSNTPF | 63 | |
| | | | TFGPGTVVDFR | | |
| 72.3 | H148- | | DYFPEPVTVSWNSGALTSG | 64 | |
| | 196 | | VHTFPAVLQSSGLYSLSSVV | | |
| | | | TVPSSSLGTK | | |
| | H219- | C223H- | YGPPCPPCPAPEFLGGPSVF | 65 | |
| | 245 | C223H | LFPPKPK | | |
| | H219- | C226H- | YGPPCPPCPAPEFLGGPSVF | 66 | |
| | 245 | C226H | LFPPKPK | | |

TABLE 3
Peptide identities of undesired major UV peaks from
acidic-pH non-reduced peptide mapping trypsin digests of
cemiplimab (FIG. 1, methods D and E). All listed peptide
sequences were confirmed using accurate full-MS and MS/MS
analysis. Unidentified peaks are presumed to be
digestive enzyme autolysis fragments.

| Retention | | Disulfide | | SEQ | |
|---|---|---|---|---|---|
| Time | Peptide | Connection | | ID | |
| (min) | Identity | Location | Peptide Sequence | NO: | Comments |
| 25.0 | N/A | | N/A | | |
| 32.2 | H66-72 | | GRFTISR | 67 | missed |
| | | | | | cleavage |
| 39.5 | N/A | | N/A | | |
| 51.3 | H358- | C364H- | NQVSLTCLVK | 68 | |
| | 367 | C422H | | | |
| | H412- | | SRWQEGNVFSCSVMHEALH | 69 | missed |
| | 436 | | NHYTQK | | cleavage |
| 52.5 | N/A | | N/A | | |
| 53.9 | H44-65 | | GLEWVSGISGGGRDTYFADS | 70 | missed |
| | | | VK | | cleavage |
| 55.1 | L127- | C134L- | SGTASVVCLLNNFYPR | 71 | |
| | 142 | C194L | | | |
| | L189- | | HKVYACEVTHQGLSSPVTK | 72 | missed |
| | 207 | | | | cleavage |
| 56.4 | H253- | C258H- | TPEVTCVVVDVSQEDPEVQF | 73 | |
| | 285 | C318H | NWYVDGVEVHNAK | | |
| | H315- | | EYKCK | 74 | missed |
| | 319 | | | | cleavage |
| 62.8 | L1-24 | C23L-C88L | DIQMTQSPSSLSASVGDSITIT | 75 | |
| | | | CR | | |
| | L77-108 | | TLQPEDFATYYCQQSSNTPF | 76 | missed |
| | | | TFGPGTVVDFRR | | cleavage |
| 63.3 | L1-24 | C23L-C88L | DIQMTQSPSSLSASVGDSITIT | 77 | |
| | | | CR | | |
| | L76-107 | | RTLQPEDFATYYCQQSSNTP | 78 | non- |
| | | | FTFGPGTVVDFR | | specific |
| | | | | | cleavage |
| 69.3 | N/A | | N/A | | trypsin |
| | | | | | autolysis |
| 70.3 | L25-61 | | ASLSINTFLNWYQQKPGKAP | 79 | missed |
| | | | NLLIYAASSLHGGVPSR | | cleavage |
| 75.3 | N/A | | N/A | | large |
| | | | | | undigested |
| | | | | | protein |
| | | | | | fragments |

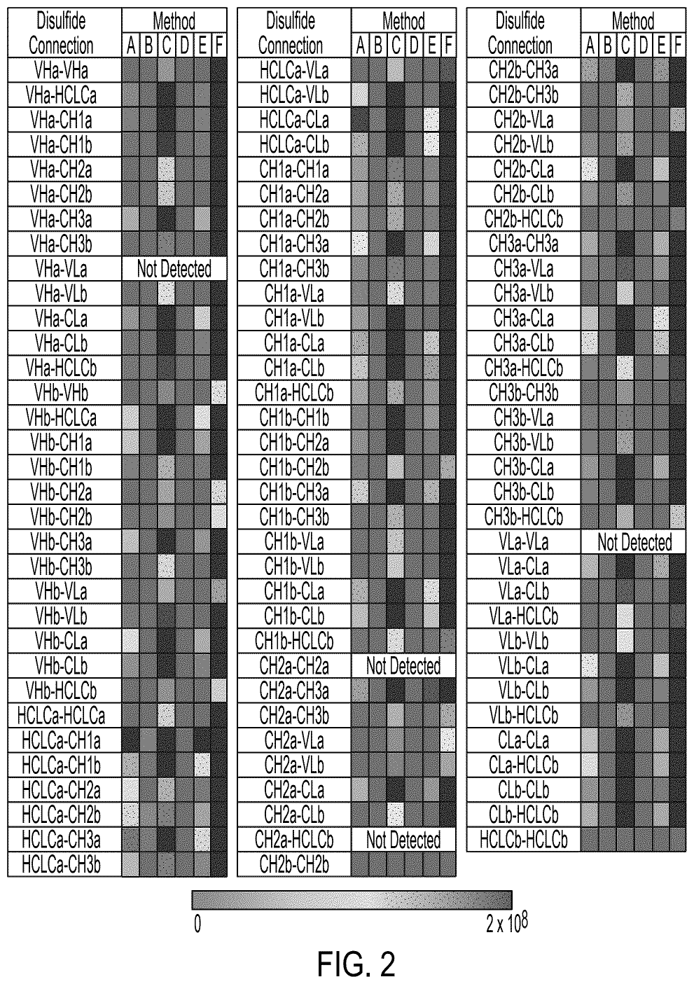

A typical homodimeric IgG4 mAb (such as cemiplimab) contains 16 unique cysteines (11 in the heavy chain and 5 in the light chain), which are usually divided among 15 unique linear peptides in a reduced trypsin digestion, with the hinge region peptide containing two cysteines ( . . . PPCPPCPA . . . ). To simplify the disulfide scrambling analysis in this study, the hinge peptide was omitted because of the complexity that each scrambled disulfide would possess as a result of the presence of multiple cysteines. The remaining 14 linear peptides (shown in the table in FIG. 2) could theoretically form disulfide connections with any other, resulting in a total of 105 unique disulfide permutations. Excluding the 7 expected native disulfide connections leaves 98 possible unique scrambled disulfides. Using the positive control digest (method C), 94 of the 98 possible peptides (95.9%) were successfully detected via MS (FIG. 2). To our knowledge, no previous study in the literature has identified such a comprehensive set of scrambled disulfides from mAb digestion.

### Example 2. Kinetics of Non-Reduced Digestion Performed With NEM

For each prepared digest, the alkylating reagent was pre-mixed into the urea denaturant solution, so that no time would elapse between the protein unfolding and availability for alkylation to occur. Therefore, only the molecular kinetics would determine whether a free thiol was alkylated and therefore deactivated from further chemistry, or was able to rearrange with other existing disulfides via thiol-disulfide exchange. Using the EICs of scrambled disulfide peptides from the positive control as references, it was simple to detect the presence of the same peptides in the other digests by matching the retention times and accurate masses. Although iodoacetamide (method A) was not expected to fully prevent scrambled disulfides from forming, an astonishing 77 of the 98 possible artifactual peptides (78.6%) were nonetheless detected, thus demonstrating that the reaction kinetics of iodoacetamide coupling with free thiols are insufficient to minimize the rearrangement of disulfide bonds.

In contrast, the basic-pH digestion performed with NEM (method B) produced only trivial MS responses for a few scrambled disulfides. Quantitatively, the average relative abundance of all scrambled disulfides for the positive control, iodoacetamide and NEM digests was 1.3%, 0.1% and 0.002%, respectively. This negligible amount indicates that NEM is extremely effective (compared to iodoacetamide) toward preventing disulfide scrambling artifacts from forming, by reacting with free thiols much faster than the rearrangement of native disulfide bonds, even under basic conditions.

In comparison, the acidic-pH digest using NEM (method D) yielded no detectable MS response for any scrambled disulfide (except a trivial amount of one peptide), as expected according to previous literature suggesting that acidic conditions would prevent such artifacts. However, unexpectedly, the acidic-pH digest with no alkylation (method E) in fact yielded substantial disulfide scrambling, with an abundance profile similar to that of the basic-pH digestion alkylated with iodoacetamide (0.1% on average for all possible peptides). Although this value was lower than that of the basic-pH digestion with no alkylation (1.3%), this indicates that digestion under acidic conditions is not the only meaningful consideration for preventing disulfide scrambling, and that the thiol-disulfide exchange mechanism still occurs if free thiols remain unalkylated. Instead, the most crucial factor is the presence of a maleimide-based alkylating reagent (e.g., NEM) because of its faster kinetics than disulfide rearrangement, even under the basic conditions optimal for trypsin digestion.

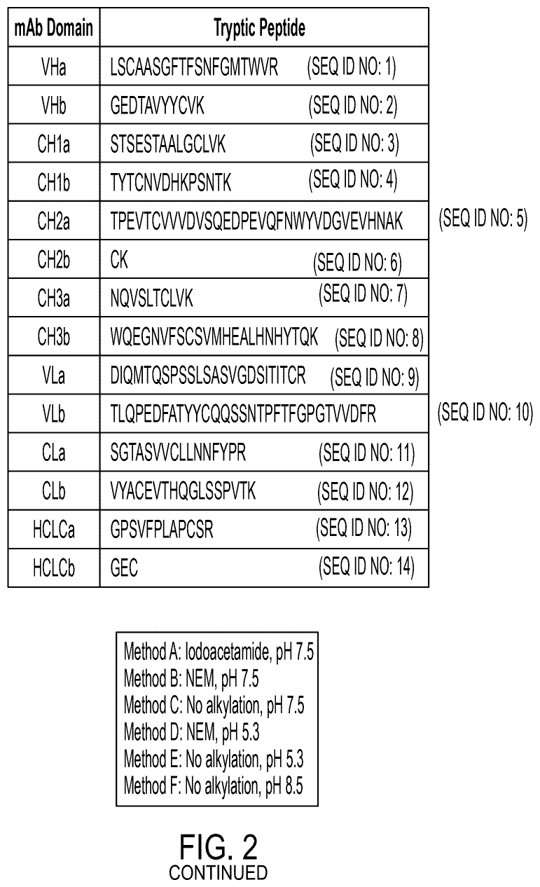

To explicitly demonstrate the difference in kinetics between iodoacetamide and NEM, two additional digests were prepared by pre-mixing alkylating reagents in the urea denaturant solution, and using the basic-pH digestion conditions as those in method A/B. The MS responses of a common IgG4 cysteine-containing peptide were extracted for both alkylated forms, as well as for an artificial scrambled disulfide involving the same linear peptide (FIG. 3). One sample contained a 1:1 mixture of each reagent at 4 mM, and the other sample contained 40 mM iodoacetamide and 2 mM NEM. The sample with the 1:1 mixture showed nearly identical results to the sample containing only NEM. In the other sample, even despite a 20-fold excess of iodoacetamide, the vast majority of the resulting alkylated peptide still existed in the NEM-labeled form, thereby demonstrating that free thiols preferentially react much faster with NEM than iodoacetamide, regardless of concentration. Additionally, the amount of the scrambled disulfide generated was minimal whenever NEM was present, and the abundance of this scrambled peptide became substantial only in the digest containing iodoacetamide alone.

### Example 3. Effect of β-Elimination on the Formation of Artificial Disulfide Scrambling

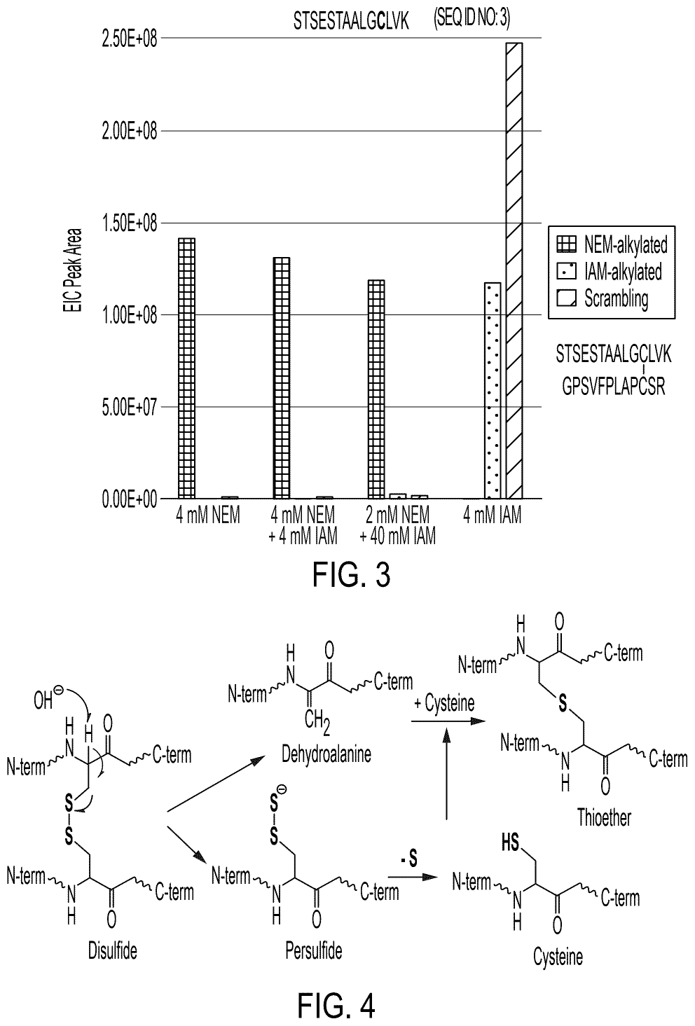

Beyond direct thiol-disulfide exchange due to the presence of pre-existing free thiols, other mechanisms can cause artificial disulfide scrambling during mAb digestion, even after free thiols have been successfully alkylated. The most commonly accepted mechanism is β-elimination, in which a hydroxide anion abstracts a proton from the carbon two atoms away (the β-position) from the sulfur of a disulfide peptide cysteine, thereby displacing the other peptide into a persulfate form, which then decomposes into free cysteine (FIG. 4). This new free thiol peptide can further exchange with a native disulfide peptide, forming a scrambled disulfide bond. Because this mechanism is initiated by hydroxide anions (—OH), it occurs more rapidly at higher pH.

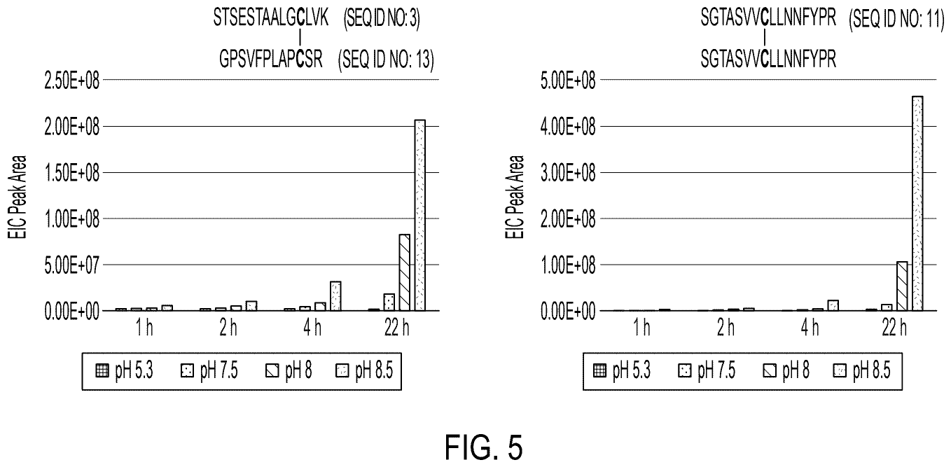

To demonstrate the effects of β-elimination on the formation of artificial disulfide scrambling, we performed a series of digests similar to method B, while varying the digestion pH and time. Each basic-pH digest was initially treated with NEM at pH 7.5 to ensure that all free thiols were quickly alkylated, and therefore any significant disulfide scrambling detected would necessarily be ascribable to mechanisms other than thiol-disulfide exchange from pre-existing free thiols. The acidic digest at pH 5.3 was prepared according to method D. A clear trend emerged in which the abundances of multiple chosen scrambled disulfides markedly increased with both digestion pH and time (FIG. 5). As expected, the acidic-pH digest yielded negligible disulfide scrambling, even after 22 h, because of the significantly lower concentration of hydroxide ions (2-3 orders of magnitude) that was present in acidic-pH digest than the basic-pH digests. In comparison, the mild conditions of method B (pH 7.5, 4 h) also yielded minor quantities of disulfide scrambling. Only under harsher conditions (e.g., pH>7.5 and time>4 h) did the effect of β-elimination significantly contribute to disulfide scrambling. Therefore, if basic conditions are chosen for a non-reduced mAb digestion, avoiding overnight incubation is recommended. Because scrambling formation due to β-elimination proceeds through continuous decomposition of native disulfide peptides during digestion, the resulting scrambled disulfide abundance is not limited by the initial free thiol quantities of the protein, as opposed to disulfide scrambling due to direct thiol-disulfide exchange.

### Example 4. Disulfide Scrambling as an Identification Tool

Once a non-reduced mAb digest has been successfully prepared with minimal artificial disulfide scrambling under our recommended conditions (method B), any significant scrambled disulfides detected can therefore be concluded to have been pre-existing in the original protein sample. One remaining challenge is that disulfide peptides can often be difficult to characterize, particularly with low MS signals, because of the potential complexity of their MS/MS fragmentation profiles. To aid in the explicit identification of each scrambled disulfide peptide, artificial scrambling formation can be maximally exploited by combining thiol-disulfide exchange and β-elimination simultaneously. A cemiplimab sample digested without alkylation and also under harsher basic conditions (method F, pH 8.5) generated a peptide mixture that contained, on average, nearly 5-fold more of each scrambled disulfide permutation than observed in the previously described positive control sample under method C (FIG. 5). Thus, with a greater MS signal, each peptide is easier to characterize with a more detailed MS/MS spectrum. Using this new positive control as a reference, any suspected real scrambled disulfides within a sample digest can be more confidently assigned.

Finally, although NEM is clearly an effective alkylating reagent for minimizing artificial disulfide scrambling during non-reduced trypsin digestion, it nonetheless possesses multiple undesired attributes. As shown in the UV chromatogram in FIG. 1, the use of NEM commonly results in two large reagent peaks (representing NEM and hydroxylated NEM), which tower above all relevant peptide peaks and are also sufficiently hydrophobic to potentially overlap with peptides, thus making the chromatogram unacceptable for therapeutic regulatory reports. In comparison, although iodoacetamide also shows a large reagent UV peak, it elutes before the peptides and therefore can be simply cropped from the displayed chromatogram. Another result of NEM's hydrophobicity, and consequently limited aqueous solubility, is that preparing a stock solution may be difficult or impossible if high concentrations are desired. Thus, an alternative alkylating reagent is recommended that provides all the same reactivity benefits of NEM without these disadvantages, yet remains unused in the literature: “maleimide”.

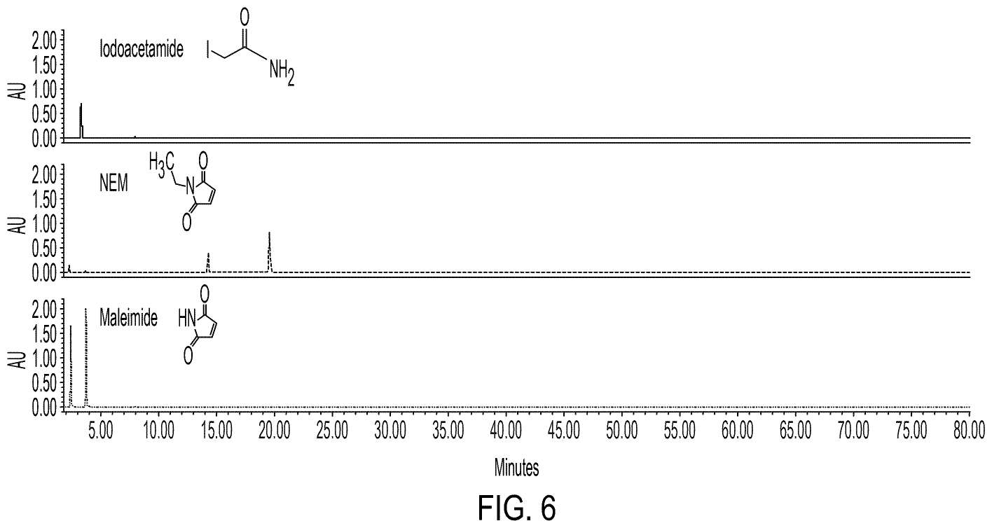

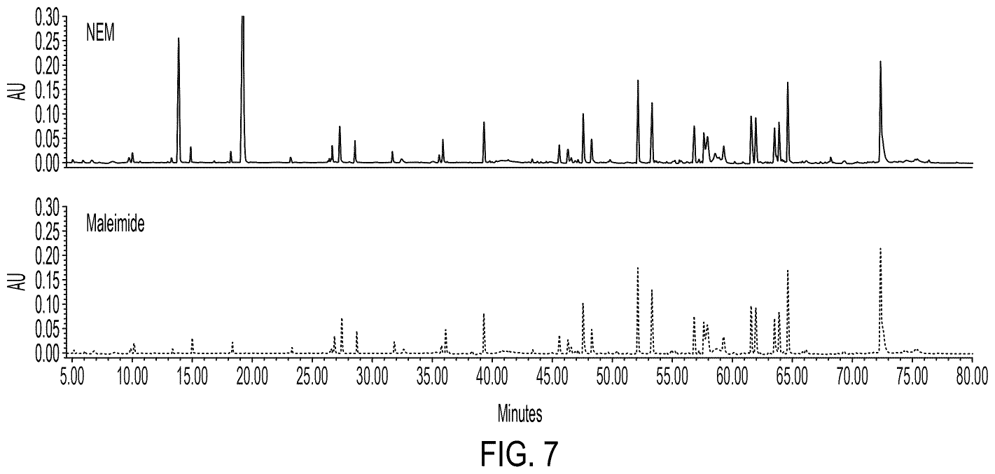

Maleimide possesses the same thiol-reactive ring structure as NEM; however, the nitrogen is substituted with hydrogen instead of an ethyl group, thus significantly decreasing its hydrophobicity. It is available commercially and inexpensively from common vendors. Maleimide has the same ability as NEM to minimize artificial disulfide scrambling during mAb digestion, analogously to method B (data not shown). Additionally, its reagent UV peaks have shorter retention times which are out of the range of reported peptides (FIG. 6 and FIG. 7), and it dissolves quickly in water at high concentrations (e.g., 0.5 M). We have observed no stability issues to date under our suggested digestion conditions (e.g., ring hydrolysis or reverse Michael addition). Maleimide may therefore be an ideal substitute for NEM for non-reduced mAb digests, while also satisfying the criteria of UV chromatograms for therapeutic regulatory reports.

The disclosure herein provides a focused and systematic approach to describe and solve common issues with non-reduced peptide mapping of mAbs. Key factors were identified to minimize disulfide scrambling artifacts while also prioritizing clean and efficient trypsin digestion under traditional basic conditions, thus eliminating the need for acidic conditions and expensive proprietary enzymes. Moreover, a method is provided to intentionally maximize disulfide scrambling as an identification tool and reference. The use of the cysteine-alkylating reagent, maleimide, is recommended, which provides all the benefits of NEM with none of the drawbacks.

All references cited herein, including U.S. patent and applications are incorporated by reference in their entirety. The present disclosure is not to be limited to the particular examples described in this application, which are intended as illustrations of various aspects. Many modifications and variations can be made without departing from its spirit and scope, as will be apparent to those skilled in the art. Functionally equivalent methods within the scope of the disclosure, in addition to those enumerated herein, will be apparent to those skilled in the art from the foregoing descriptions. Such modifications and variations are intended to fall within the scope of the appended claims.

What is claimed is:

1. A method for performing non-reduced peptide mapping of a protein of interest, comprising:
(a) alkylating the protein of interest with N-ethyl maleimide (NEM) or an NEM analog under denaturing conditions to form an alkylated protein of interest;
(b) digesting the alkylated protein of interest with at least one digestive enzyme to form a peptide digest; and
(c) analyzing the peptide digest by liquid chromatography-mass spectrometry to obtain a non-reduced peptide mapping of the protein of interest;
wherein digesting the alkylated protein of interest is carried out at a pH from about 6.5 to about 8.3.

2. The method of claim 1, wherein step (a) comprises premixing a denaturing agent the N-ethyl maleimide (NEM) or the NEM analog to form a premixture and adding the protein of interest to the premixture.

3. The method of claim 1, further comprising identifying disulfide bonds in the protein of interest.

4. The method of claim 1, wherein the protein of interest is an antibody, a bispecific antibody, a monoclonal antibody, a fusion protein, an antibody-drug conjugate, an antibody fragment, or a protein pharmaceutical product.

5. The method of claim 1, wherein said NEM analog is maleimide.

6. The method of claim 1, wherein the denaturing conditions include use of urea, guanidinium chloride, an organic solvent, or a combination thereof.

7. The method of claim 1, wherein the at least one digestive enzyme includes an immunoglobulin G-degrading enzyme of Streptococcus pyogenes, sialidase, cysteine protease, endopeptidase, papain, endoproteinase Lys-C, pepsin, trypsin, carboxypeptidase B, protease, or a combination thereof.

8. The method of claim 1, wherein the at least one digestive enzyme includes trypsin.

9. The method of claim 1, wherein alkylating the protein of interest is carried out at a pH from about 6.5 to about 8.

10. The method of claim 1, wherein the digesting is conducted at a pH from about 7.0 to about 8.0.

11. The method of claim 1, wherein the digesting is conducted at a pH of 7.5±0.2.

12. The method of claim 1, wherein the at least one digestive enzyme includes trypsin; wherein alkylating the protein of interest is carried out at a pH from about 6.5 to about 8; and wherein the digesting is conducted at a pH from about 7 to about 8.

13. The method of claim 1, wherein liquid chromatography comprises reversed phase liquid chromatography, ion exchange chromatography, size exclusion chromatography, affinity chromatography, hydrophobic interaction chromatography, hydrophilic interaction chromatography, mixed-mode chromatography, or a combination thereof.

14. The method of claim 1, wherein analyzing the peptide digest by liquid chromatography-mass spectrometry includes analysis with an liquid chromatography system coupled to a mass spectrometer.

15. The method of claim 14, wherein the mass spectrometer is an electrospray ionization mass spectrometer, nano-electrospray ionization mass spectrometer, quadrupole mass spectrometer, an Orbitrap-based mass spectrometer, or a hybrid quadrupole-Orbitrap-based mass spectrometer.

16. The method of claim 14, wherein the mass spectrometer is a tandem mass spectrometry.

17. The method of claim 1, wherein an amino acid is added to an eluate between the liquid chromatography and the mass spectrometry.

18. The method of claim 17, wherein the amino acid is glycine.


SEQUENCE LISTING (14 sequences)

SEQ ID NO: 1 - protein, 19 aa, synthetic construct
LSCAASGFTFSNFGMTWVR

SEQ ID NO: 2 - protein, 11 aa, synthetic construct
GEDTAVYYCVK

SEQ ID NO: 3 - protein, 14 aa, synthetic construct
STSESTAALGCLVK

SEQ ID NO: 4 - protein, 14 aa, synthetic construct
TYTCNVDHKPSNTK

SEQ ID NO: 5 - protein, 33 aa, synthetic construct
TPEVTCVVVDVSQEDPEVQFNWYVDGVEVHNAK

SEQ ID NO: 6 - skipped

SEQ ID NO: 7 - protein, 10 aa, synthetic construct
NQVSLTCLVK

SEQ ID NO: 8 - protein, 23 aa, synthetic construct
WQEGNVFSCSVMHEALHNHYTQK

SEQ ID NO: 9 - protein, 24 aa, synthetic construct
DIQMTQSPSSLSASVGDSITITCR

SEQ ID NO: 10 - protein, 31 aa, synthetic construct
TLQPEDFATYYCQQSSNTPFTFGPGTVVDFR

SEQ ID NO: 11 - protein, 16 aa, synthetic construct
SGTASVVCLLNNFYPR

SEQ ID NO: 12 - protein, 17 aa, synthetic construct
VYACEVTHQGLSSPVTK

SEQ ID NO: 13 - protein, 12 aa, synthetic construct
GPSVFPLAPCSR

SEQ ID NO: 14 - skipped